(19) United States
(12) Patent Application Publication
(10) Pub. No.: US 2025/0222040 A1
(43) Pub. Date: Jul. 10, 2025

(54) PHARMACEUTICAL COMPOSITION AND USE THEREOF IN PREPARATION OF DRUG FOR TREATING CHRONIC KIDNEY DISEASE

(71) Applicant: Union Hospital Tongji Medical College HUST, Wuhan (CN)

(72) Inventors: Chun Zhang, Wuhan (CN); Jianshe Liu, Wuhan (CN)

(21) Appl. No.: 18/404,623

(22) Filed: Jan. 4, 2024

(30) Foreign Application Priority Data
May 19, 2023 (CN) 202310587510.2

(51) Int. Cl. A61K 35/64; A61K 35/62; A61K 36/481; A61K 36/708; A61P 13/12

(52) U.S. Cl. CPC A61K 35/64; A61K 35/62; A61K 36/481; A61K 36/708; A61P 13/12; A61K 2236/15; A61K 2236/331; A61K 2236/333; A61K 2236/39

(57) ABSTRACT

Provided is a pharmaceutical composition and use thereof in treating a chronic kidney disease. In the pharmaceutical composition, Astragalus membranaceus is adopted for strengthening body resistance, and Radix et Rhizoma Rhei, Whitmania pigra (W. pigra), and Bombyx batryticatus (B. batryticatus) are adopted for eliminating pathogens, that is, the pharmaceutical composition can improve a kidney function through the combination of strengthening body resistance with eliminating pathogens. The above treatments all can significantly reduce creatinine, urea nitrogen, and urine protein/creatinine ratio indexes in rats with chronic renal failure (CRF), and improve pathological changes such as glomerular mesangial expansion, extracellular matrix deposition of mesangial cells, glomerulosclerosis, and renal interstitial fibrosis (RIF) in rats (a model) with CRF, thereby further improving a therapeutic effect of the TCM composition for CKD.

### CROSS-REFERENCE TO RELATED APPLICATION

This patent application claims the benefit and priority of Chinese Patent Application No. 202310587510.2 filed with the China National Intellectual Property Administration on May 19, 2023, the disclosure of which is incorporated by reference herein in its entirety as part of the present application.

### TECHNICAL FIELD

The present disclosure belongs to the technical field of traditional Chinese medicine (TCM), and specifically relates to a pharmaceutical composition and a use thereof in preparation of a drug for treating a chronic kidney disease (CKD).

### BACKGROUND

CKD refers to a chronic progressive irreversible damage to a structure or function of kidneys caused by various factors. Since CKD is irreversible, early intervention and the development of a method to delay disease progression are particularly important to solve the dilemma of CKD treatment. It is believed in TCM that CKD is a syndrome of deficiency in origin and excess in superficiality, and is manifested as the deficiency of qi and blood in viscera, the dampness turbidity, and the retention of stasis and toxins; and CKD is treated mainly by replenishing deficiency, dispelling dampness, detoxifying, and promoting blood circulation and removing blood stasis.

There is a long history of using TCM for treating kidney diseases with definite therapeutic effects, where rich experiences have been accumulated and relevant TCM theoretical guidance is provided. For example, in the journal paper “Observation of Clinical Efficacy of Shenkang Injection in Treatment of Chronic Kidney Failure” (Fan Jun, Journal of Medical Information (Mid-Term), Issue 2, 2011), Radix et Rhizoma Rhei, Astragalus membranaceus, Salvia miltiorrhiza (S. miltiorrhiza), and Carthamus tinctorius L. are used to prepare the Shenkang injection. Studies have shown that the Shenkang injection can delay glomerulosclerosis and renal interstitial fibrosis (RIF), and effectively reduce a blood creatinine level and a blood urea nitrogen (BUN) level and improve a creatinine clearance rate for a patient to ultimately improve a glomerular filtration rate (GFR) of the patient, thereby allowing the purpose of CKD treatment. In the Master's thesis “Clinical Study of Shenshuai Yangzhen Granules in Improvement of Malnutrition of Stage 4 and 5 CKD” (Zhang Ying), the Shenshuai Yangzhen (nourishing the deficient kidney) granules are prepared with Astragalus membranaceus, Angelica sinensis, ginseng, Atractylodes macrocephala, Radix et Rhizoma Rhei, and Amomum villosum (A. villosum) as main components to treat patients with stage 4 and 5 CKD. Studies have shown that the Shenshuai Yangzhen granules can improve the malnutrition and anemia conditions of patients with CKD 4 and 5.

However, in the prior art, most attention is paid to the optimization of raw materials for a TCM formula, and the influence of a corresponding preparation process or specific active ingredients is not considered. In particular, most TCM formulas are decocted with water and then administered; or a TCM formula is first subjected to water extraction, and then resulting water-soluble active ingredients are collected and prepared into different dosage forms for administration, where merely water-soluble active ingredients (which are mainly water-soluble polysaccharides (WSPs)) are extracted. TCM also includes active ingredients such as fat-soluble flavones and alkaloids, which also exhibit specified therapeutic effects. In addition, biological activities of many polysaccharides are related to molecular weights, solubility, viscosity, degrees of branching (DBs), chain conformations, and crystallinity of these polysaccharides, and this relationship between structure and biological activity of polysaccharides is called a structure-activity relationship (SAR) of polysaccharides. Therefore, different active ingredients can be extracted from a TCM formula and then water-soluble ingredients can be further purified to obtain polysaccharides of specific molecular weights and purities, which can affect the activity of the TCM formula.

The present disclosure adopts Astragalus membranaceus, Radix et Rhizoma Rhei, Bombyx batryticatus (B. Batryticatus), and Whitmania pigra (W. pigra) in combination. Astragalus membranaceus is a sovereign TCM component with a function of invigorating qi and protecting kidneys, and enters the lung and spleen meridians. Astragalus membranaceus has the effects of replenishing qi and consolidating the exterior, promoting urination and expelling toxicity, discharging pus, and promoting granulation and wound healing. Radix et Rhizoma Rhei has always been known for attacking stagnation, clearing damp heat, purging fire, cooling blood, eliminating stasis, and detoxifying. Astragalus membranaceus and Radix et Rhizoma Rhei play an upfloating role and a downsinking role, respectively, and can closely cooperate with each other to treat closed sweat pore and unclean bladder conditions of many edema diseases caused by the fact that lucid yang fails to rise and turbid yin fails to fall. However, in addition to the above two manners, the treatment of an edema requires “eliminating depressed stale water”, namely, “dissolving stasis and phlegm to remove stagnation”, and thus W. pigra (leech) is adopted as an adjuvant component to promote blood circulation and remove blood stasis. W. pigra is a component for promoting blood circulation and removing blood stasis. Modern studies have shown that hirudin has anti-coagulation and anti-thrombosis effects, and can increase blood flows in kidneys to improve kidney functions. A main function of W. pigra is to remove blood stasis and resolve masses. Radix et Rhizoma Rhei also has an effect of promoting blood circulation and removing blood stasis. B. batryticatus is also adopted as an adjuvant component for dissolving phlegm, eliminating wind pathogens, and regulating meridians. W. pigra and B. batryticatus both are worm drugs, and have a strong effect of eliminating scurried pathogens. With reference to the Shengjiang powder in the “Detailed Discrimination between Typhoid Fever and Seasonal Febrile Disease”, the compatibility of B. batryticatus with Radix et Rhizoma Rhei is taken to play a role of ascending lucidity and descending turbidity. The combination of the above four TCM components can exhibit effects of tonifying qi and strengthening body resistance, ascending lucidity and descending turbidity, promoting blood circulation, dissolving phlegm, and inducing urination, which is conducive to the treatment of qi deficiency, blood stasis, phlegm obstruction, and water retention common in many kidney diseases. In the TCM composition of the present disclosure, Astragalus membranaceus is adopted for strengthening body resistance, and Radix et Rhizoma Rhei, W. pigra, and B. batryticatus are adopted for eliminating pathogens, that is, the TCM composition can improve a kidney function through the combination of strengthening body resistance with eliminating pathogens.

On the basis of the above formula, the present disclosure further investigates a preparation process of the TCM composition and therapeutic effects of related fat-soluble active components to determine the influence of the preparation process and different fat-soluble active components on a therapeutic effect for a CKD.

### SUMMARY

In view of the problems in the prior art, the present disclosure provides a pharmaceutical composition and a use thereof in preparation of a drug for treating a CKD. In the present disclosure, based on a formula including Astragalus membranaceus, Radix et Rhizoma Rhei, B. batryticatus, and W. pigra, a decoction solution obtained after decocting W. pigra, B. batryticatus, and Radix et Rhizoma Rhei in water is subjected to dialysis to remove small-molecule polysaccharides and impurities; and/or a residue obtained after decocting W. pigra, B. batryticatus, and Radix et Rhizoma Rhei in water is further subjected to alcohol extraction to obtain active ingredients such as flavones, and the active ingredients are used for the TCM composition, which can improve a therapeutic effect of the TCM composition for CKD.

To achieve the above objective, in a first aspect, the present disclosure provides a TCM composition prepared from the following raw materials in parts by weight: 45 parts to 55 parts of Astragalus membranaceus, 8.5 parts to 11.5 parts of Radix et Rhizoma Rhei, 5 parts to 7 parts of W. pigra, and 5 parts to 7 parts of B. batryticatus, where Radix et Rhizoma Rhei includes 2.5 parts to 3.5 parts of Radix et Rhizoma Rhei for pulverization and 6 parts to 8 parts of Radix et Rhizoma Rhei for extraction; and the TCM composition is prepared as follows: subjecting W. pigra, B. batryticatus, and Radix et Rhizoma Rhei for extraction to extraction with a solvent and then to concentration to obtain a concentrate, mixing and pulverizing Astragalus membranaceus and Radix et Rhizoma Rhei for pulverization to obtain a Astragalus membranaceus/Radix et Rhizoma Rhei mixed powder, and mixing the concentrate with the Astragalus membranaceus/Radix et Rhizoma Rhei mixed powder, where the solvent is water and/or an ethanol aqueous solution.

In a preferred embodiment, a process of subjecting W. pigra, B. batryticatus, and Radix et Rhizoma Rhei for extraction to the extraction with water is as follows:
(1) adding W. pigra and B. batryticatus to water, decocting, and filtering to obtain a first extract solution and a first filter residue; and
(2) adding Radix et Rhizoma Rhei for extraction to the first filter residue, adding water, decocting, and filtering to obtain a second extract solution and a second filter residue.

In a preferred embodiment, a process of subjecting W. pigra, B. batryticatus, and Radix et Rhizoma Rhei for extraction to the extraction with water and then with the ethanol aqueous solution is as follows:
(1) adding W. pigra and B. batryticatus to water, decocting, and filtering to obtain a first extract solution and a first filter residue;
(2) adding Radix et Rhizoma Rhei for extraction to the first filter residue, adding water, decocting, and filtering to obtain a second extract solution and a second filter residue; and
(3) adding the ethanol aqueous solution to the second filter residue obtained in step (2) to allow extraction, and filtering to obtain a third extract solution; and combining the first extract solution, the second extract solution, and the third extract solution, and allowing ethanol to be completely volatilized.

In a preferred embodiment, after W. pigra, B. batryticatus, and Radix et Rhizoma Rhei for extraction are subjected to the extraction with water, a dialysis step is further included; and a specific process of the dialysis step is as follows: conducting dialysis for 36 h to 72 h in a dialysis bag with a molecular weight cut-off (MWCO) of 6,000 to 9,000, and collecting retentate in the dialysis bag.

In a preferred embodiment, a volume fraction of ethanol in the ethanol aqueous solution is 60% to 85% by weight.

In a preferred embodiment, when the ethanol aqueous solution is used for extraction, the ethanol aqueous solution is added in 150 to 350 parts by weight, and the extraction is conducted at 50° C. to 70° C. for 2 h to 4 h.

In a preferred embodiment, for decoction each time, water is added in 160 to 300 parts by weight, and the decoction is conducted for 30 min to 90 min.

In a preferred embodiment, a process of mixing and pulverizing Astragalus membranaceus and Radix et Rhizoma Rhei for pulverization is as follows: mixing and pulverizing Astragalus membranaceus and Radix et Rhizoma Rhei for pulverization, and sieving a pulverized material through a 50- to 200-mesh sieve to obtain the Astragalus membranaceus/Radix et Rhizoma Rhei mixed powder.

In a preferred embodiment, the concentration is conducted under reduced pressure until a density of the concentrate is 1.12 to 1.15.

In a second aspect, the present disclosure provides the use of the TCM composition in preparation of a drug for treating a CKD.

In a preferred embodiment, a dosage form of the drug is selected from the group consisting of a pill, a tablet, and a granule; and a preparation process of the pill includes: subjecting the TCM composition to sieving, bulking, shaping, coating, drying, pill screening, quality inspection, and packaging.

The present disclosure has the following beneficial effects:

1. In the TCM composition of the present disclosure, Astragalus membranaceus is adopted for strengthening body resistance, and Radix et Rhizoma Rhei, W. pigra, and B. batryticatus are adopted for eliminating pathogens, that is, the TCM composition can improve kidney function through the combination of strengthening body resistance with eliminating pathogens.

2. In the present disclosure, a decoction solution obtained after decocting W. pigra, B. batryticatus, and Radix et Rhizoma Rhei in water is subjected to dialysis to remove small-molecule polysaccharides and impurities; and a residue obtained after decocting W. pigra, B. batryticatus, and Radix et Rhizoma Rhei in water is further subjected to alcohol extraction to obtain fat-soluble active ingredients such as flavones, and the fat-soluble active ingredients are used for the TCM composition. All of the above treatments can significantly reduce creatinine, urea nitrogen, and urine protein/creatinine ratio indexes in rats with chronic renal failure (CRF) and improve pathological changes such as glomerular mesangial expansion, mesangial matrix deposition, glomerulosclerosis, and RIF in rats (a model) with CRF, thereby further improving a therapeutic effect of the TCM composition for CKD.

### BRIEF DESCRIPTION OF THE DRAWINGS

FIG. 1 shows serum Scr contents, BUN contents, and total urine protein/creatinine ratio indexes of test rats in examples and a comparative example;

FIG. 2 shows Periodic Acid-Schiff (PAS) staining results of Examples 1 to 3 and Comparative Example 1;

FIG. 3 shows a PAS staining result of Example 4;

FIG. 4 shows Masson staining results of Examples 1 to 3 and Comparative Example 1; and

FIG. 5 shows a Masson staining result of Example 4.

### DETAILED DESCRIPTION OF THE EMBODIMENTS

It should be noted that the raw materials used in the present disclosure are ordinary commercially available products, and sources of the raw materials are not specifically limited.

### Example 1

A formula of a TCM composition was as follows: 50 g of Astragalus membranaceus, 10 g of Radix et Rhizoma Rhei (7 g of Radix et Rhizoma Rhei for extraction+3 g of Radix et Rhizoma Rhei for pulverization), 6 g of W. pigra, and 6 g of B. batryticatus.

A preparation process of the TCM composition was as follows:
(1) Astragalus membranaceus and Radix et Rhizoma Rhei for pulverization were mixed and pulverized, and then sieved through an 80-mesh sieve to obtain an Astragalus membranaceus/Radix et Rhizoma Rhei mixed powder.
(2) W. pigra and B. batryticatus were added to 200 mL of water, and a resulting mixture was decocted for 45 min and then filtered to obtain a first extract solution and a first filter residue.
(3) Radix et Rhizoma Rhei for extraction was added to the first filter residue, then 200 mL of water was added, and a resulting mixture was decocted for 45 min and then filtered to obtain a second extract solution and a second filter residue.
(4) 200 mL of ethanol aqueous solution with a volume fraction of 75% was added to the second filter residue obtained in step (3) to allow for extraction at 60° C. for 3 h, and a resulting system was filtered to obtain a third extract solution.
(5) The first extract solution obtained in step (2), the second extract solution obtained in step (3), and the third extract solution obtained in step (4) were combined, and the combined extract solution was evaporated to remove ethanol completely and then concentrated under reduced pressure to obtain a concentrate with a density of 1.12 to 1.15; and then the concentrate was mixed with the Astragalus membranaceus/Radix et Rhizoma Rhei mixed powder obtained in step (1), and the resulting mixture was subjected to sieving, bulking, shaping, coating, drying, pill screening, quality inspection, and packaging.

### Example 2

A formula of a TCM composition was as follows: 50 g of Astragalus membranaceus, 10 g of Radix et Rhizoma Rhei (7 g of Radix et Rhizoma Rhei for extraction+3 g of Radix et Rhizoma Rhei for pulverization), 6 g of W. pigra, and 6 g of B. batryticatus.

A preparation process of the TCM composition was as follows:
(1) Astragalus membranaceus and Radix et Rhizoma Rhei for pulverization were mixed and pulverized, and then sieved through an 80-mesh sieve to obtain an Astragalus membranaceus/Radix et Rhizoma Rhei mixed powder.
(2) W. pigra and B. batryticatus were added to 200 mL of water, and a resulting mixture was decocted for 45 min and then filtered to obtain a first extract solution and a first filter residue.
(3) Radix et Rhizoma Rhei for extraction was added to the first filter residue, then 200 mL of water was added, and a resulting mixture was decocted for 45 min and then filtered to obtain a second extract solution and a second filter residue.
(4) The first extract solution obtained in step (2) and the second extract solution obtained in step (3) were combined, a combined extract solution was subjected to dialysis for 48 h in a dialysis bag with a molecular weight cutoff (MWCO) of 7,000, and retentate in the dialysis bag was collected and concentrated under reduced pressure to obtain a concentrate with a density of 1.12 to 1.15; and then the concentrate was mixed with the Astragalus membranaceus/Radix et Rhizoma Rhei mixed powder obtained in step (1), and a resulting mixture was subjected to sieving, bulking, shaping, coating, drying, pill screening, quality inspection, and packaging.

### Example 3

A formula of a TCM composition was as follows: 50 g of Astragalus membranaceus, 10 g of Radix et Rhizoma Rhei (7 g of Radix et Rhizoma Rhei for extraction+3 g of Radix et Rhizoma Rhei for pulverization), 6 g of W. pigra, and 6 g of B. batryticatus.

A preparation process of the TCM composition was as follows:
(1) Astragalus membranaceus and Radix et Rhizoma Rhei for pulverization were mixed and pulverized, and then sieved through an 80-mesh sieve to obtain an Astragalus membranaceus/Radix et Rhizoma Rhei mixed powder.
(2) W. pigra and B. batryticatus were added to 200 mL of water, and a resulting mixture was decocted for 45 min and then filtered to obtain a first extract solution and a first filter residue.
(3) Radix et Rhizoma Rhei for extraction was added to the first filter residue, then 200 mL of water was added, and a resulting mixture was decocted for 45 min and then filtered to obtain a second extract solution and a second filter residue.
(4) 200 mL of ethanol aqueous solution with a volume fraction of 75% was added to the second filter residue obtained in step (3) to allow for extraction at 60° C. for 3 h, and a resulting system was filtered to obtain a third extract solution.
(5) The first extract solution obtained in step (2) and the second extract solution obtained in step (3) were combined, a combined extract solution was subjected to dialysis for 48 h in a dialysis bag with an MWCO of 7,000, and retentate in the dialysis bag was collected; the third extract solution obtained in step (4) was added to the retentate, and a resulting mixed solution was evaporated to remove ethanol completely and then concentrated under reduced pressure to obtain a concentrate with a density of 1.12 to 1.15; and then the concentrate was mixed with the Astragalus membranaceus/Radix et Rhizoma Rhei mixed powder obtained in step (1), and the resulting mixture was subjected to sieving, bulking, shaping, coating, drying, pill screening, quality inspection, and packaging.

### Example 4

A formula of a TCM composition was as follows: 55 g of Astragalus membranaceus, 11.5 g of Radix et Rhizoma Rhei (8 g of Radix et Rhizoma Rhei for extraction+3.5 g of Radix et Rhizoma Rhei for pulverization), 7 g of W. pigra, and 7 g of B. batryticatus.

A preparation process of the TCM composition was as follows:
(1) Astragalus membranaceus and Radix et Rhizoma Rhei for pulverization were mixed and pulverized, and then sieved through an 80-mesh sieve to obtain an Astragalus membranaceus/Radix et Rhizoma Rhei mixed powder.
(2) W. pigra and B. batryticatus were added to 250 ml of water, and a resulting mixture was decocted for 60 min and then filtered to obtain a first extract solution and a first filter residue.
(3) Radix et Rhizoma Rhei for extraction was added to the first filter residue, then 250 mL of water was added, and a resulting mixture was decocted for 60 min and then filtered to obtain a second extract solution and a second filter residue.
(4) 250 mL of an ethanol aqueous solution with a volume fraction of 75% was added to the second filter residue obtained in step (3) to allow extraction at 70° C. for 2.5 h, and a resulting system was filtered to obtain a third extract solution.
(5) The first extract solution obtained in step (2) and the second extract solution obtained in step (3) were combined, a combined extract solution was subjected to dialysis for 48 h in a dialysis bag with an MWCO of 8,000, and retentate in the dialysis bag was collected; the third extract solution obtained in step (4) was added to the retentate, and a resulting mixed solution was evaporated to remove ethanol completely and then concentrated under reduced pressure to obtain a concentrate with a density of 1.12 to 1.15; and then the concentrate was mixed with the Astragalus membranaceus/Radix et Rhizoma Rhei mixed powder obtained in step (1), and the resulting mixture was subjected to sieving, bulking, shaping, coating, drying, pill screening, quality inspection, and packaging.

### Comparative Example 1

A formula of a TCM composition was as follows: 50 g of Astragalus membranaceus, 10 g of Radix et Rhizoma Rhei (7 g of Radix et Rhizoma Rhei for extraction+3 g of Radix et Rhizoma Rhei for pulverization), 6 g of W. pigra, and 6 g of B. batryticatus.

A preparation process of the TCM composition was as follows:
(1) Astragalus membranaceus and Radix et Rhizoma Rhei for pulverization were mixed and pulverized, and then sieved through an 80-mesh sieve to obtain an Astragalus membranaceus/Radix et Rhizoma Rhei mixed powder.
(2) W. pigra and B. batryticatus were added to 200 mL of water, and a resulting mixture was decocted for 45 min and then filtered to obtain a first extract solution and a first filter residue.
(3) Radix et Rhizoma Rhei for extraction was added to the first filter residue, then 200 mL of water was added, and a resulting mixture was decocted for 45 min and then filtered to obtain a second extract solution.
(4) The first extract solution obtained in step (2) and the second extract solution obtained in step (3) were combined, and the combined extract solution was concentrated under reduced pressure to obtain a concentrate with a density of 1.12 to 1.15; and then the concentrate was mixed with the Astragalus membranaceus/Radix et Rhizoma Rhei mixed powder obtained in step (1), and the resulting mixture was subjected to sieving, bulking, shaping, coating, drying, pill screening, quality inspection, and packaging.

### Experimental Example

Properties of the TCM compositions obtained in Examples 1 to 4 and Comparative Example 1 were verified.

### 1. Modeling and Drug Administration

42 male SD rats were randomly divided into 7 groups with 6 rats per group, including a normal group (namely, a control group), a model group, example groups 1 to 4, and a comparative example group 1 (namely, a comparative example group). Rats in the groups other than the control group each were subjected to 5/6 nephrectomy to obtain CRF rat models. After successful modeling, the CRF rat models each were treated. The drug administration was started 2 weeks after the 5/6 nephrectomy and conducted continuously for 8 weeks, and then samples were collected, where a blood sample was collected by removing eyeballs and a urine sample was collected by forced urination. The administration mode for the example groups 1 to 4 and the comparative example group was as follows: The TCM compositions of Examples 1 to 4 and Comparative Example 1 each were ground into powder, the powder was dissolved in normal saline (NS) to obtain a 0.5 g/mL suspension, and the suspension was intragastrically injected into a rat at a dose of 2 g/kg/d. In the model group, normal saline was intragastrically injected at the same dose.

### 2. Detection of Kidney Functions of Rats

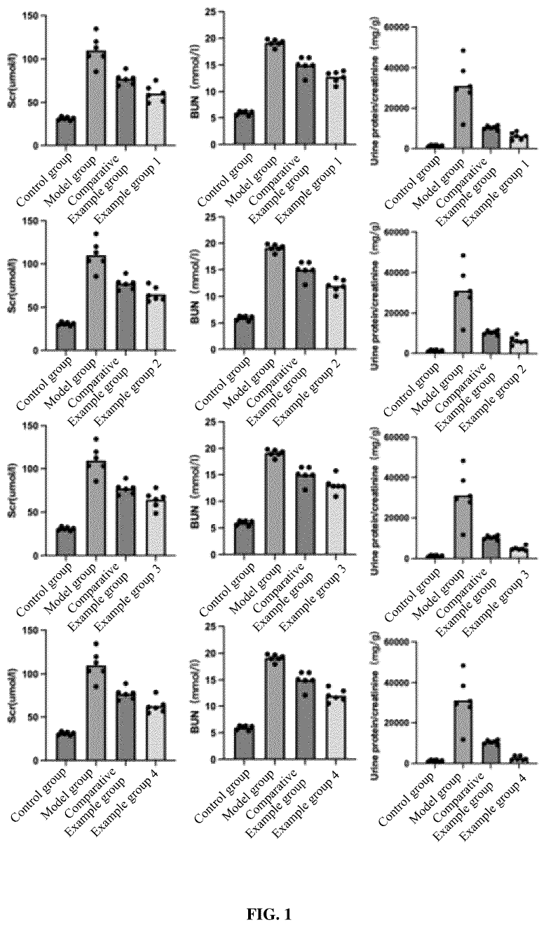

For each rat in each group, a blood sample was collected and centrifuged to obtain serum, and a urine sample was collected by forced urination; and renal function-associated indexes (creatinine, urea nitrogen, and urine protein/creatinine ratio) in serum and urine were detected by an automatic biochemical detector, and results are shown in FIG. 1.

It can be seen from FIG. 1 that after intervention with the TCM compositions of Examples 1 to 4 and Comparative Example 1, the creatinine, urea nitrogen, and urine protein/creatinine ratio indexes in CRF rats decreased significantly, where the creatinine, urea nitrogen, and urine protein/creatinine ratio indexes decreased more significantly in the example groups 1 to 4 than in the comparative example group 1, indicating that in the example groups 1 to 4, residual renal functions could be well protected, complications were reduced, and the CKD progression was delayed.

### 3. Detection of the Influence of the TCM Compositions on Disease Progression in CRF Rats

Kidney tissue sections were stained by PAS staining and Masson staining to determine the influence of the TCM compositions on disease progression in CRF rats of the example groups 1 to 4 and the comparative example group 1.

### 3.1 PAS Staining

PAS staining (Periodic Acid Staining) is mainly used to detect saccharides in a tissue histologically. Periodic acid can oxidize hydroxyl groups on two adjacent carbons of a saccharide into an aldehyde group, and then a Schiff's reagent is allowed to react with the aldehyde group to allow the presentation of a purplish-red color. Ethylene glycol (EG) groups in glycogen or polysaccharide substances (such as mucopolysaccharides, mucoproteins, glycoproteins, and glycolipids) present in a cytoplasm can be oxidized by periodic acid into a dialdehyde group, and the dialdehyde group can be combined with colorless magenta in the Schiff's reagent to produce a purplish-red dye deposited at a location of intracellular polysaccharides. This reaction is called a PAS positive reaction and was also previously known as glucogen staining.

### Main Reagents

(1) Periodic acid oxidation solution: 1 g of HIO₄·2H₂O was taken, then distilled water was added to 100 mL, and after the solid was completely dissolved, a resulting solution was stored in a refrigerator for later use.

### (2) Schiff's Reagent:

1 g of magenta was dissolved in 200 mL of boiled distilled water, and the resulting solution was shaken for 5 min, cooled to about 60° C., and filtered to obtain a first filtrate; 20 mL of 1 mol/L HCl was added to the first filtrate, and the resulting mixed solution was thoroughly mixed and cooled to 25° C.; 2 g of sodium metabisulfite (Na₂S₂O₅) or potassium metabisulfite (K₂S₂O₅) was added, and the resulting mixture was filled in a brown glass bottle with a stopper and placed in the dark for 24 h; and 1 g of activated carbon was added, the resulting mixture was shaken for 1 min and filtered through a filter paper to obtain a second filtrate, and the second filtrate was stored in a refrigerator.

### (3) Sulfurous Acid Solution:

The sulfurous acid solution was freshly prepared with 6 mL of a 100 g/L sodium metabisulfite solution, 5 mL of a 1 mol/L hydrochloric acid solution, and 100 mL of distilled water just before use.

### (4) 20 g/L Methyl Green Solution:

2 g of methyl green was taken, and distilled water was added to 100 mL.

(5) Amylase solutions: The secretion of saliva was stimulated by chewing paraffin, and saliva was collected and centrifuged to obtain an amylase-containing supernatant; and 0.1 g to 1.0 g of maltogenic amyiase was dissolved in 100 mL of 0.02 mol/L phosphate buffered saline (PBS) (pH 6.0).

### PAS Staining Steps

(1) A fresh and dry smear was fixed with 5% (w/w) ethanol for 10 min, then rinsed with distilled water, and naturally dried.

(2) If the smear required digestion, saliva or maltogenic amyiase could be added to allow for digestion at room temperature for 60 min, and then the smear was rinsed with distilled water.

(3) The smear was oxidized with 10 g/L periodic acid for 15 min to 20 min, then rinsed with distilled water, and naturally dried.

(4) The smear was stained at room temperature in a Schiff's staining solution for 30 min to 60 min.

(5) The smear was rinsed 3 times with the sulfurous acid solution, then rinsed with tap water for 2 min to 3 min, and naturally dried.

(6) The smear was counterstained with the 20 g/L methyl green solution for 10 min to 20 min.

(7) The smear was washed with water, naturally dried, and subjected to microscopic examination.

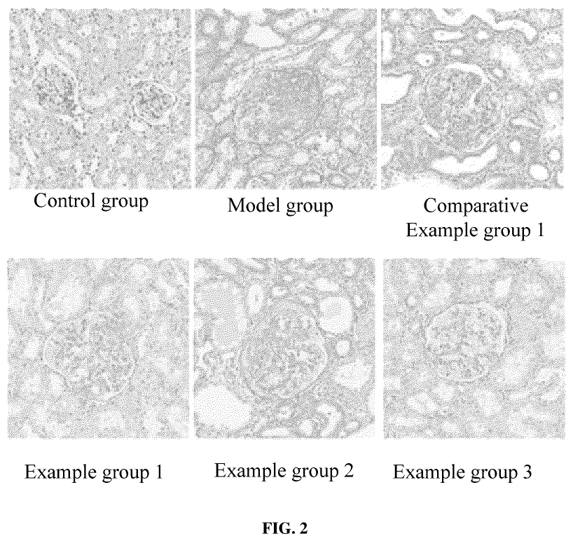

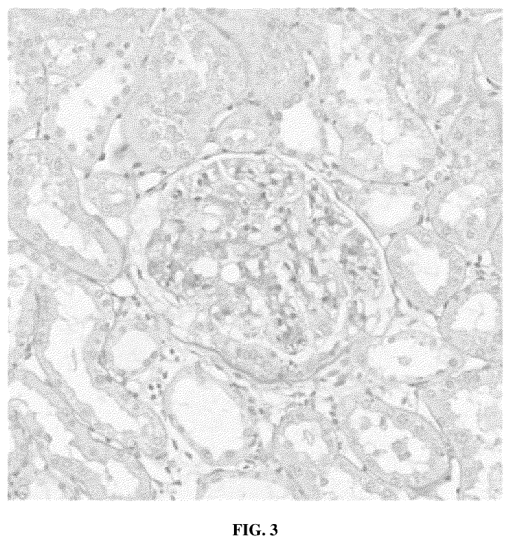

PAS staining results of the example groups 1 to 4 and the comparative example group 1 are shown in FIG. 2 and FIG. 3. It can be seen from the PAS staining results in FIG. 2 and FIG. 3 that the CRF rats (models) underwent glomerular mesangial expansion, significant mesangial matrix deposition, and significant glomerulosclerosis, and the above pathological changes could be effectively improved in the example groups 1 to 4 and the comparative example 1, where the pathological changes were improved more significantly in the example groups 1 to 4 than in the comparative example 1.

### 3.2 Masson Staining

Masson staining is one of the staining methods to display fibers in a tissue. The Masson staining is related to a size of an anionic dye molecule and the permeability of a tissue. The size of the anionic dye molecule is reflected by a molecular weight. An anionic dye molecule with a small molecular weight is easy to penetrate through a tissue with a dense structure and low permeability; and an anionic dye molecule with a large molecular weight can only enter a tissue with a loose structure and high permeability. Light green SF or aniline blue has a large molecular weight. Therefore, after Masson staining, muscle fibers turn red and collagenous fibers turn green or blue.

### Masson Staining Steps

(1) Dewaxation of paraffin section: A paraffin section was dewaxed with first xylene for 5 min, second xylene for 5 min, third xylene for 5 min, absolute ethanol for 1 min, 95% ethanol for 1 min, and 75% ethanol for 1 min, and then rinsed with tap water for a few seconds.

(2) Chromation or mercury salt precipitate removal was conducted (this step could be omitted for a formaldehyde-fixed tissue).

(3) The section was washed with tap water and distilled water successively.

(4) The section was subjected to nuclear staining with a Regaud hematoxylin staining solution or a Weigert iron hematoxylin staining solution for 5 min to 10 min.

(5) The section was differentiated with 1% hydrochloric acid in an alcohol for 5 s to 15 s, and then fully washed with water.

(6) A 0.1% to 1% lithium carbonate solution was added dropwise for 5 min to increase an anti-blue degree, and then the section was washed with water.

(7) The section was stained with a Masson ponceau acid fuchsin solution for 5 min to 10 min.

(8) The section was washed with a 2% glacial acetic acid aqueous solution for 1 min.

(9) The section was differentiated with a 1% phosphomolybdic acid aqueous solution for 3 min to 5 min and then washed with a 2% glacial acetic acid aqueous solution for 1 min.

(10) Without water ashing, the section was directly stained with an aniline blue aqueous solution or a 1% glossy green aqueous solution for 1 min to 2 min, and then washed with a 0.2% glacial acetic acid aqueous solution for 1 min.

(11) The section was dehydrated with 95% ethanol for 2 s to 3 s and then with absolute ethanol 3 times for 5 s each time, permeabilized with xylene 3 times for 1 min each time, and then mounted with a neutral gum.

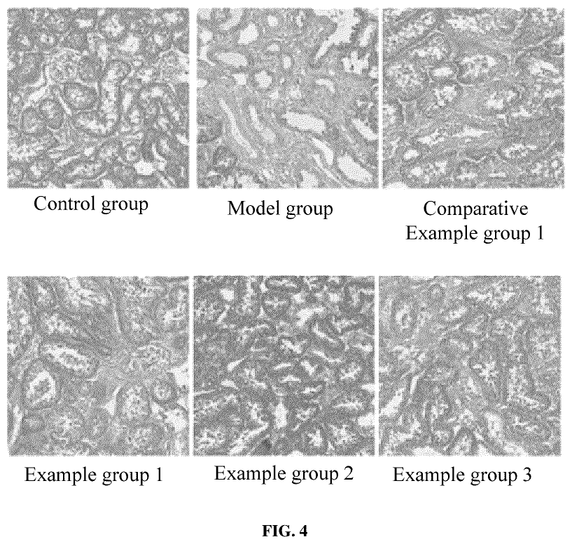

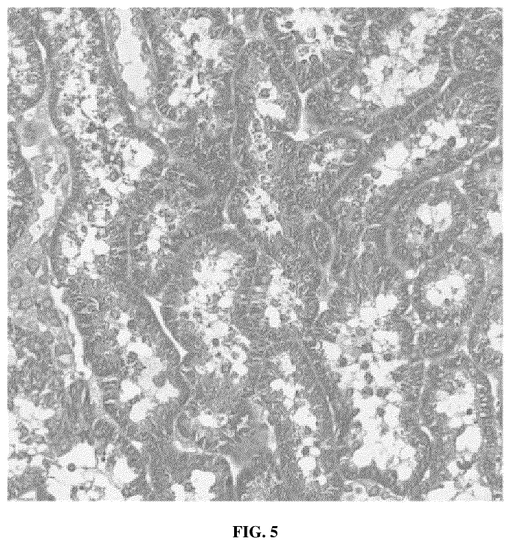

After staining, collagenous fibers or collagen, mucus, and cartilages were blue (they were green if a glossy green solution was used for staining); cytoplasm, muscle, cellulose, glia, and erythrocytes were red; and nuclei were blue-purple. Masson staining results of the example groups 1 to 4 and the comparative example group 1 are shown in FIG. 4 and FIG. 5.

It can be seen from the Masson staining results in FIG. 4 and FIG. 5 that the CRF rats (model group) underwent proximal tubular brush border disappearance, renal tubular dilatation, inflammatory cell infiltration, and significant RIF (blue), and the above pathological changes can be effectively improved in the example groups 1 to 4 and the comparative example 1, where the pathological changes were improved more significantly in the example groups 1 to 4 than in the comparative example 1.

In the present disclosure, a decoction solution obtained after decocting W. pigra, B. batryticatus, and Radix et Rhizoma Rhei in water is subjected to dialysis to remove small-molecule polysaccharides and impurities; and a residue obtained after decocting W. pigra, B. batryticatus, and Radix et Rhizoma Rhei in water is further subjected to alcohol extraction to obtain fat-soluble active ingredients such as flavones, and the fat-soluble active ingredients are used for the TCM composition. The above treatments all can significantly reduce creatinine, urea nitrogen, and urine protein/creatinine ratio indexes in rats with CRF, and improve pathological changes such as glomerular mesangial expansion, mesangial matrix deposition, glomerulosclerosis, and RIF in rats (a model) with CRF, thereby further improving a therapeutic effect of the TCM composition for CKD.

The solutions of the present disclosure are not limited to the technical means disclosed by the above technical means, and further include technical solutions composed of any combination of the above technical features. The above are preferred implementations of the present disclosure. It should be noted that various improvements and modifications can be made by a person of ordinary skill in the art without departing from the principles of the present disclosure, and these improvements and modifications should also be regarded as falling within the protection scope of the present disclosure.

What is claimed is:

1. A traditional Chinese medicine (TCM) composition comprising raw materials in parts by weight: 45 parts to 55 parts of Astragalus membranaceus, 8.5 parts to 11.5 parts of Radix et Rhizoma Rhei, 5 parts to 7 parts of Whitmania pigra (W. pigra), and 5 parts to 7 parts of Bombyx batryticatus (B. batryticatus), wherein Radix et Rhizoma Rhei comprises 2.5 parts to 3.5 parts of Radix et Rhizoma Rhei for pulverization and 6 parts to 8 parts of Radix et Rhizoma Rhei for extraction; and the TCM composition is prepared as follows: subjecting W. pigra, B. batryticatus, and Radix et Rhizoma Rhei for extraction to extraction with a solvent and then to concentration to obtain a concentrate, mixing and pulverizing Astragalus membranaceus and Radix et Rhizoma Rhei for pulverization to obtain a Astragalus membranaceus/Radix et Rhizoma Rhei mixed powder, and mixing the concentrate with the Astragalus membranaceus/Radix et Rhizoma Rhei mixed powder, wherein the solvent is water and/or an ethanol aqueous solution.

2. The TCM composition according to claim 1, wherein a process of subjecting W. pigra, B. batryticatus, and Radix et Rhizoma Rhei for extraction to the extraction with water is as follows:
(1) adding W. pigra and B. batryticatus to water, decocting, and filtering to obtain a first extract solution and a first filter residue; and
(2) adding Radix et Rhizoma Rhei for extraction to the first filter residue, adding water, decocting, and filtering to obtain a second extract solution and a second filter residue; and
combining the first extract solution and the second extract solution.

3. The TCM composition according to claim 1, wherein a process of subjecting W. pigra, B. batryticatus, and Radix et Rhizoma Rhei for extraction to the extraction with water and the ethanol aqueous solution is as follows:
(1) adding W. pigra and B. batryticatus to water, decocting, and filtering to obtain a first extract solution and a first filter residue;
(2) adding Radix et Rhizoma Rhei for extraction to the first filter residue, adding water, decocting, and filtering to obtain a second extract solution and a second filter residue; and
(3) adding the ethanol aqueous solution to the second filter residue obtained in step (2) to allow extraction, and filtering to obtain a third extract solution; and combining the first extract solution, the second extract solution, and the third extract solution, and allowing ethanol to be completely volatilized.

4. The TCM composition according to claim 1, wherein after W. pigra, B. batryticatus, and Radix et Rhizoma Rhei for extraction are subjected to the extraction with water, a dialysis step is further comprised.

5. The TCM composition according to claim 2, wherein after W. pigra, B. batryticatus, and Radix et Rhizoma Rhei for extraction are subjected to the extraction with water, a dialysis step is further comprised.

6. The TCM composition according to claim 3, wherein after W. pigra, B. batryticatus, and Radix et Rhizoma Rhei for extraction are subjected to the extraction with water, a dialysis step is further comprised.

7. The TCM composition according to claim 4, wherein a specific process of the dialysis step is as follows: conducting dialysis for 36 h to 72 h in a dialysis bag with a molecular weight cut-off (MWCO) of 6,000 to 9,000, and collecting retentate in the dialysis bag.

8. The TCM composition according to claim 1, wherein a volume fraction of ethanol in the ethanol aqueous solution is 60% to 85%; when the ethanol aqueous solution is used for extraction, the ethanol aqueous solution is added in 150 to 350 parts by weight, and the extraction is conducted at 50° C. to 70° C. for 2 h to 4 h; and when decoction is conducted, water is added in 160 to 300 parts by weight, and the decoction is conducted for 30 min to 90 min.

9. The TCM composition according to claim 1, wherein a process of mixing and pulverizing Astragalus membranaceus and Radix et Rhizoma Rhei for pulverization is as follows: mixing and pulverizing Astragalus membranaceus and Radix et Rhizoma Rhei for pulverization, and sieving a pulverized material through a 50 to 200-mesh sieve to obtain a Astragalus membranaceus/Radix et Rhizoma Rhei mixed powder.

10. The TCM composition according to claim 1, wherein the concentration is conducted under reduced pressure until a density of the concentrate is 1.12 to 1.15.

11. A method for treating a chronic kidney disease (CKD), comprising administering a medicament comprising the TCM composition according to claim 1 to a subject in need thereof.

12. The method according to claim 11, wherein a dosage form of the medicament is selected from the group consisting of a pill, a tablet, and a granule; and a preparation process of the pill comprises: subjecting the TCM composition to sieving, bulking, shaping, coating, drying, pill screening, quality inspection, and packaging.
